(19) United States
(12) Patent Application Publication
(10) Pub. No.: US 2025/0051835 A1
(43) Pub. Date: Feb. 13, 2025

(54) SIGNAL NORMALIZATION OF NUCLEIC ACID AMPLIFICATION REACTION PRODUCTS

(71) Applicant: QIAGEN GMBH, Hilden (DE)

(72) Inventor: Stefan Otto Cornelius, Hilden (DE)

(21) Appl. No.: 18/717,806

(22) PCT Filed: Oct. 17, 2022
(86) PCT No.: PCT/EP2022/078868

§ 371 Date: Jun. 7, 2024

(30) Foreign Application Priority Data
Dec. 8, 2021 (EP) 21213242.7

(51) Int. Cl. C12Q 1/6844

(52) U.S. Cl. CPC C12Q 1/6844

(57) ABSTRACT

The invention relates to a multiplex nucleic acid amplification reaction comprising at least two separate amplicons, with normalized signal strength, wherein for at least one amplicon either the forward primer oligonucleotides, or the reverse primers oligonucleotides, or the optional detection probe oligonucleotides, are only partially labeled, and the subset amount of labeled primer/probe oligonucleotides to subset amount of unlabeled primer/probe oligonucleotides is adjusted so that the signal strength in both amplicons is equal or nearly equal. Kits and compositions are also claimed.

### FIELD OF THE INVENTION

The present invention is in the field of molecular biology, in particular in the field of normalization of the signal strength of nucleic acid amplification reaction products. It is also in the field of molecular genetics and more particular in the field of genotyping, forensics, molecular diagnostics and population genetics.

### BACKGROUND OF THE INVENTION

The invention relates to methods, compositions and kits for the normalization of the signal strength of one or more signals of nucleic acid amplification reaction products without the need for a quantification of template nucleic acid concentrations prior to the amplification reaction. Also included in the present invention are methods, compositions and kits for genotyping and forensic applications.

The most commonly used technique in molecular biology and molecular diagnostics is the polymerase chain reaction (PCR) which is the basic technique for nearly all modern biological research and molecular diagnostic applications such as diagnosis for infectious diseases, forensics, human identification, paternity testing, population genetics, cancer diagnostics and many more.

Several workflow schemes in the above mentioned application fields entail the amplification of so called short tandem repeat (STR) markers, also referred to as microsatellites or simple sequence repeats (SSR). These markers are genetic elements of variable lengths which are characterized by short repetitive sequence motifs. Usually, a set of different STR markers, i.e. different loci, are analyzed for example in order to obtain a genetic fingerprint of an individual. This information can be further used e.g. in forensics to accurately identify or eliminate a suspect by comparing this fingerprint with evidence from a crime scene, in paternity testing, in population genetics, genotyping of the human leucocyte antigen (HLA) genes and many more.

Classically, after amplification in a PCR, the resulting PCR products were usually labeled with intercalating dyes, such as ethidium bromide or SYBR Green and analyzed after separation in gel electrophoresis. In principle, this procedure is still commonly used. With the development of modern nucleic acid extraction methods, improved PCR techniques including improved polymerases and buffers and an overall robust PCR chemistry, PCR thermocyclers, fluorescent dyes and also with the development of highly sensitive capillary electrophorese (CE) methods and apparatuses, the workflow became even more relevant and essential in standard applications as described above.

A modern workflow for STR marker analysis usually comprises multiplex PCR amplification techniques which allow for the simultaneous amplification of several STR markers in the same reaction while incorporating fluorescently labeled primers and/or probes with different fluorophores for each STR marker. In the subsequent CE, the PCR products, i.e. the amplified and differentially labeled STR markers, are separated according to their size and detected by the fluorescence signals which are represented as peaks in the electropherogram of the CE. These peaks are usually automatically detected and scored by a software.

Despite all the developments and improvements which were made over the last decades, this workflow still requires accurately quantified concentrations of the template nucleic acids to be analyzed. In case of a too high input sample concentration in the PCR and/or in the subsequent CE, the capillaries will be overloaded and/or the fluorescence signal is too high and the STR profile cannot be determined (c.f. FIG. 1b). In such cases the sample must be diluted to a lower concentration. In case of a too low input sample concentration, the fluorescence signal is too low and the STR profile cannot be determined either (c.f. FIG. 1c). The whole workflow as described above is well established and described in the related scientific literature (e.g. Barbaro A. (2018) Forensic Genetics, In: Barbaro A (2018) Manual of Forensic Science: An International Survey. CRC Press, 289 pages, ISBN 978-1-4987-6630-2).

Thus, an accurate nucleic acid concentration quantification is usually required. Several methods and kits are available on the market for quantitative PCR (qPCR) based quantification (e.g.: QIAGEN Investigator Quantiplex Pro Kit, catalogue number 387216; ThermoFisher Scientific/Applied Biosystems Quantifiler Trio DNA Quantification Kit, catalogue number 4482910; Promega PowerQuant System, catalogue number PQ5008). However, the quantification means an additional step in the overall workflow which requires additional laboratory time, equipment and personnel. Furthermore, every additional step in the overall workflow increases the risk of handling errors, sample contamination and sample loss.

Therefore, there is an urgent need for a system that allows the researcher and laboratory personnel to avoid the quantification step, sample concentration calculation and sample dilution steps in the overall workflow and thus to reduce the costs, turn-around-time and the risk of sample contamination and sample loss.

### SUMMARY OF THE INVENTION

In order to avoid the essential quantification step in current STR analysis or genotyping assays, the inventor developed methods, compositions and kits for the normalization of the signal strength of nucleic acid amplification reaction products, e.g. for STR analysis, genotyping or molecular diagnostics, which work with a wide range of template nucleic acid concentrations without the risk of fluorescence signal detection errors in the CE and thus without the need for the steps of nucleic acid quantification, concentration calculation and dilution. Therefore, the invention increases the robustness of current STR analysis, genotyping assays and molecular diagnostic assays, workflows and kits, reduces the assay costs and reduces the turn-around-time and the risk for contamination and/or sample loss dramatically.

The invention relates to a multiplex nucleic acid amplification reaction comprising at least two separate amplicons, with normalized signal strength, wherein for at least one amplicon either the forward primer oligonucleotides, or the reverse primers oligonucleotides, or the optional detection probe oligonucleotides, are only partially labeled, and the subset amount of labeled primer/probe oligonucleotides to subset amount of unlabeled primer/probe oligonucleotides is adjusted so that the signal strength in both amplicons is equal or nearly equal.

The invention relates to a method for normalizing the signal strength of one or more signals of nucleic acid amplification reaction products comprising the steps of (a) providing a nucleic acid amplification reaction mixture comprising one or more template nucleic acids, said template nucleic acids comprising one or more target nucleic acid sequences, and one or more sets of oligonucleotides, wherein each set of oligonucleotides comprises one or more oligonucleotides, wherein at least one of said oligonucleotides is hybridizable to at least one of said target nucleic acid sequences, wherein each of said sets of oligonucleotides comprises at least (i) a forward primer, (ii) a revers primer and/or (iii) a probe or any combination of (i) to (iii) above, and wherein at least one of the hybridizable oligonucleotides selected from (i) to (iii) above comprises a first subset which is labeled with one or more detectable labels and a second subset which is unlabeled, (b) performing a nucleic acid amplification reaction and (c) detecting the one or more signals of the one or more detectable labels of the first labeled subset of said at least one hybridizable oligonucleotide, wherein the signal strength of the one or more detected signals is normalized by the ratio of the first labeled subset and the second unlabeled subset of said at least one hybridizable oligonucleotide.

The method according to the invention can be performed in a singleplex amplification reaction as well as in a multiplex amplification reaction comprising several different sets of oligonucleotides.

The invention further relates to a composition comprising two or more sets of oligonucleotides, wherein each of said sets of oligonucleotides comprises at least
(i) a forward primer,
(ii) a revers primer, and/or
(iii) a probe, and
wherein at least one of the oligonucleotides selected from (i) to (iii) above comprises a first subset which is labeled with one or more detectable labels and a second subset which is unlabeled.

The invention further relates to a kit comprising one or more sets of oligonucleotides, wherein each of said sets of
oligonucleotides comprises at least
   (i) a forward primer,
   (ii) a revers primer, and optionally
   (iii) a probe, and
wherein at least one of the oligonucleotides selected from (i) to (iii) above comprises a first subset of oligonucleotides which is labeled with one or more detectable labels and a second subset of oligonucleotides which is unlabeled.

### DETAILED DESCRIPTION OF THE INVENTION

The invention relates to methods, compositions and kits for the normalization of the signal strength of one or more signals of nucleic acid amplification reaction products without the need for quantification of template nucleic acid concentrations prior to the amplification. The methods, compositions and kits allow for a broader range of template nucleic acid amounts used in the amplification reactions and lead to a more robust and accurate analysis of the amplification reaction products. The invention further relates to methods, compositions and kits for the normalization of the signal strength of one or more signals of nucleic acid amplification reaction products without the need for the steps of concentration calculation and dilution prior to the amplification reaction.

The invention further relates to a multiplex nucleic acid amplification reaction comprising at least two separate amplicons, with normalized signal strength, wherein for at least one amplicon either the forward primer oligonucleotides, or the reverse primers oligonucleotides, or the optional detection probe oligonucleotides, are only partially labeled, and the subset amount of labeled primer/probe oligonucleotides to subset amount of unlabeled primer/probe oligonucleotides is adjusted so that the signal strength in both amplicons is equal or nearly equal.

The invention further relates to a nucleic acid amplification reaction comprising at least one amplicon, with adjusted signal strength, wherein for at least said one amplicon either the forward primer oligonucleotides, or the reverse primers oligonucleotides, or the optional detection probe oligonucleotides, are only partially labeled, and the subset amount of labeled primer/probe oligonucleotides to subset amount of unlabeled primer/probe oligonucleotides is adjustable.

Adjustability is used to adjust for template amount. Hence, if the template is clean and abundant one might use a low ratio of labelled primer/probe to unlabeled primer/probe, in contrast if the template is less abundant one might raise the ration of labeled to unlabeled primer/probe.

Such single reactions might be used to analyze difficult forensic samples with varying amounts of template nucleic acids.

The invention further relates to a composition comprising two or more sets of oligonucleotides, wherein each of said sets of oligonucleotides comprises at least
(i) a forward primer,
(ii) a revers primer, and/or
(iii) a probe, and
   wherein at least one of the oligonucleotides selected from (i) to (iii) above comprises a first subset which is labeled with one or more detectable labels and a second subset which is unlabeled.

The invention further relates to a kit comprising one or more sets of oligonucleotides, wherein each of said sets of oligonucleotides comprises at least
(i) a forward primer,
(ii) a revers primer, and optionally
(iii) a probe, and
   wherein at least one of the oligonucleotides selected from (i) to (iii) above comprises a first subset of oligonucleotides which is labeled with one or more detectable labels and a second subset of oligonucleotides which is unlabeled.

In the reaction ideally, the ratio of the labeled subset to unlabeled subset is between 100:1 and 1:10000, even more preferably is between 10:1 and 1:5000, even more preferably is between 1:1 and 1:400, even more preferably is between 1:1 and 300, even more preferably is between 1:1 and 200, even more preferably is between 1:1 and 100, even more preferably is between 1:5 and 500, even more preferably between 1:5 and 1:200, even more preferably between 1:5 and 1:100 and most preferably is between 1:10 and 1:100.

In the reaction ideally, the plateau phase of the amplification reaction is reached.

In the reaction according to the invention preferably the amplicons are located on one or more template nucleic acids and the amount of the one or more template nucleic acids in said nucleic acid amplification reaction mixture is between 1 fg to 5 pg, more preferably between 100 fg to 1 pg, even more preferably between 500 fg to 500 ng, even more preferably between 750 fg to 250 ng, even more preferably between 850 fg to 250 ng, even more preferably between 900 fg to 150 ng, even more preferably between 1 μg to 150 ng and most preferably between 2 μg to 110 ng.

Preferably, no quantification and/or dilution of the one or more template nucleic acids is performed prior to the amplification reaction. This is in fact a benefit of the present invention.

Preferably, detecting the labels of the first subset of oligonucleotides is performed in capillary electrophoresis, gel electrophoresis, pyrosequencing, sanger sequencing, next generation sequencing, digital PCR, real-time PCR, quantitative PCR, isothermal PCR, or in microarray analysis.

And it is very preferred, if the reaction is an endpoint PCR, a digital PCR, a real-time PCR, a quantitative PCR, an isothermal PCR, a loop-mediated isothermal amplification, a recombinase polymerase amplification, a nicking enzyme amplification reaction, a nicking endonuclease signal amplification, a rolling circle amplification, a helicase-dependent amplification, a hybridization chain reaction, a multidisplacement amplification, an isothermal assembly reaction or any combination thereof.

Ideally and preferably, the one or more template nucleic acids are extracted from a forensic sample containing biological material, sputum, saliva, blood, hairs, hair follicles, sperm, vaginal secretions, liquor, blood plasma, blood serum, fingernails, tissue, urine, plants, microbes, bacteria, viruses, any other parts of the human or animal body or from any other sample containing biological material from which nucleic acids can be extracted.

The samples are preferably forensic samples. Preferably, they are contaminated with other DNA from third party individuals or inhibitors.

The reaction is preferably for use in STR analysis, SNP analysis, genotyping, molecular diagnostics, genetic research or population genetics.

Most preferably the reaction is a STR analysis reaction.

The invention also relates to a composition comprising two or more sets of oligonucleotides, wherein each of said sets of oligonucleotides comprises at least
(i) a forward primer,
(ii) a revers primer, and/or
(iii) a probe, and
   wherein at least one of the oligonucleotides selected from (i) to (iii) above comprises a first subset which is labeled with one or more detectable labels and a second subset which is unlabeled.

It also relates to a composition, wherein the ratio of the first labeled subset and the second unlabeled subset of said at least one of the oligonucleotides is between 100:1 and 1:10000, even more preferably is between 10:1 and 1:5000, even more preferably is between 1:1 and 1:400, even more preferably is between 1:1 and 300, even more preferably is between 1:1 and 200, even more preferably is between 1:1 and 100, even more preferably is between 1:5 and 500, even more preferably between 1:5 and 1:200, even more preferably between 1:5 and 1:100 and most preferably is between 1:10 and 1:100.

The invention likewise relates to a kit comprising one or more sets of oligonucleotides, wherein each of said sets of
oligonucleotides comprises at least
   (i) a forward primer,
   (ii) a revers primer, and optionally
   (iii) a probe, and
      wherein at least one of the oligonucleotides selected from (i) to (iii) above comprises a first subset of oligonucleotides which is labeled with one or more detectable labels and a second subset of oligonucleotides which is unlabeled.

Here, the ratio of the first labeled subset and the second unlabeled subset of oligonucleotides is between 100:1 and 1:10000, even more preferably is between 10:1 and 1:5000, even more preferably is between 1:1 and 1:400, even more preferably is between 1:1 and 300, even more preferably is between 1:1 and 200, even more preferably is between 1:1 and 100, even more preferably is between 1:5 and 500, even more preferably between 1:5 and 1:200, even more preferably between 1:5 and 1:100 and most preferably is between 1:10 and 1:100.

The method/reaction according to this invention can be applied to several assays and analyzing methods such as—but not limited to—STR analysis and genotyping, nucleic acid library preparation, digital PCR and molecular diagnostics.

The method/reaction according to this invention showed several improvements over the methods that are known in the art. The STR analysis kits which are currently commercially available (e.g.: Promega VersaPlex 27PY System, catalogue number DC7020; Thermo Fisher Scientific/Applied Biosystems Yfiler Plus PCR Amplification Kit; catalogue number 4484678; QIAGEN Investigator 26plex QS Kit, catalogue number 382615) require for an accurate quantification of the template nucleic acids, the calculation of sample volumes and most often for sample dilution. Compared to the methods known in the art, the method according to the invention does not require these time-intensive, cost-intensive and lab personnel intensive steps. Therefore, it significantly reduces the turn-around-time, assay costs and reduces the risk for sample cross contamination.

The STR analysis methods known in the art are very sensitive to the amount of template nucleic acids. Therefore an accurate quantification step as well as the steps of template nucleic acid calculation and dilution are currently mandatory and usually explicitly mentioned in the kit handbooks. FIG. 1 shows the importance of the above mentioned mandatory steps in currently available STR analysis kits. While the assays are optimized and quite robust for a specific amount of template nucleic acids which is usually about 500 pg (c.f. FIG. 1a), the kits and methods are error prone if the amount of template nucleic acids is above (c.f. FIG. 1b) or below (c.f. FIG. 1c) the recommended range of the amount of template nucleic acids.

Current commercially available STR PCR kits contain sets of oligonucleotides wherein at least one of the oligonucleotides, e.g. the forward primer, the revers primer or a probe, is completely labeled with a detectable label. Unexpectedly, the inventor found out during his extensive research that limiting the amount of labeled oligonucleotides while not limiting the overall amount of said oligonucleotides, i.e. providing a first subset of at least one of the oligonucleotides labeled with a detectable label and a second subset of said at least one of the oligonucleotides which is unlabeled, leads to a better and more robust performance of the STR assays in terms of significantly broader ranges of template nucleic acid amounts which can be directly used in the PCR and in terms of stable fluorescence signals in the CE.

The invention also relates to a method for normalizing the signal strength of a signal of nucleic acid amplification reaction products comprising the steps of (a) providing a nucleic acid amplification reaction mixture comprising a template nucleic acid, said template nucleic acid comprising a target nucleic acid sequence, and a set of oligonucleotides, wherein the set of oligonucleotides comprises one or more oligonucleotides, wherein at least one of said oligonucleotides is hybridizable to said target nucleic acid sequence, wherein said set of oligonucleotides comprises at least (i) a forward primer, (ii) a revers primer, and/or (iii) a probe or any combination of (i) to (iii) above, and wherein at least one of the hybridizable oligonucleotides selected from (i) to (iii) above comprises a first subset which is labeled with one or more detectable labels and a second subset which is unlabeled, (b) performing a nucleic acid amplification reaction, (c) detecting the one or more signals of the one or more detectable labels of the first labeled subset of said at least one hybridizable oligonucleotide, wherein the signal strength of the one or more detected signals is normalized by the ratio of the first labeled subset and the second unlabeled subset of at least one hybridizable oligonucleotide.

Multiplex amplification is a well-established optimization of the amplification techniques known in the art. It allows for the simultaneous amplification of several loci in one amplification reaction and the simultaneous or subsequent detection and analysis of the amplification reaction products by differentiation of the amplicon lengths and/or different detectable labels for the different loci in the same amplification reaction or amplification reaction products, respectively. Due to differentially labeled primers and/or probes, the loci can even be overlapping. The method according to the invention can also be performed as multiplex amplification reaction.

Thus, in a preferred embodiment the invention relates to a method for normalizing the signal strength of one or more signals of nucleic acid amplification reaction products comprising the steps of (a) providing a nucleic acid amplification reaction mixture comprising one or more template nucleic acids, said template nucleic acids comprising one or more target nucleic acid sequences, and one or more sets of oligonucleotides, wherein each set of oligonucleotides comprises one or more oligonucleotides, wherein at least one of said oligonucleotides is hybridizable to at least one of said target nucleic acid sequences, wherein each of said sets of oligonucleotides comprises at least (i) a forward primer, (ii) a revers primer, and/or (iii) a probe or any combination of (i) to (iii) above, and wherein at least one of the hybridizable oligonucleotides selected from (i) to (iii) above comprises a first subset which is labeled with one or more detectable labels and a second subset which is unlabeled, (b) performing a nucleic acid amplification reaction, (c) detecting the one or more signals of the one or more detectable labels of the first labeled subset of said at least one hybridizable oligonucleotide, wherein the signal strength of the one or more detected signals is normalized by the ratio of the first labeled subset and the second unlabeled subset of said at least one hybridizable oligonucleotide.

In another embodiment the invention relates to a composition comprising one or more sets of oligonucleotides, wherein each of said sets of oligonucleotides comprises at least (i) a forward primer, (ii) a revers primer, and/or (iii) a probe or any combination of (i) to (iii) above, and wherein at least one of the oligonucleotides selected from (i) to (iii) above comprises a first subset which is labeled with one or more detectable labels and a second subset which is unlabeled.

In a further embodiment, the invention relates to a kit comprising one or more sets of oligonucleotides, wherein each of said sets of oligonucleotides comprises at least (i) a forward primer, (ii) a revers primer, and/or (iii) a probe or any combination of (i) to (iii) above, and wherein at least one of the oligonucleotides selected from (i) to (iii) above comprises a first subset which is labeled with one or more detectable labels and a second subset which is unlabeled.

Herein, “normalization” is defined as the process of equalizing the template nucleic acid concentration prior to amplification and/or equalizing the detection signal of the nucleic acid amplification products to a range in which the signal strength is optimal for a reliable detection and/or quantification of the nucleic acid amplification products. Equalizing the template nucleic acid concentration usually requires the determination of the template nucleic acid concentration and a further step of diluting it to an optimal template nucleic acid concentration for the nucleic acid amplification reaction. However, in cases where the template nucleic acid concentration is too low, this equalization of the template nucleic acid concentration is not possible. Equalizing the detection signal of the nucleic acid amplification products for the subsequent amplification reaction according to the invention is performed by limiting the amount of the detectable label of one or more oligonucleotides in a set of oligonucleotides which are incorporated into the nucleic acid amplification products during amplification. This avoids a signal strength which is above the optimal range for a proper detection and/or quantification which is important in cases where the template nucleic acid concentration is too high. Additionally, the amplification reaction may be performed until the plateau phase is reached which ensures that an optimal signal strength can be detected even at low template nucleic acid concentrations.

In one embodiment, the signal strength of one or more signals of nucleic acid amplification reaction products is normalized by the limitation of the amount of labeled oligonucleotides while not limiting the overall amount of said oligonucleotides. This means that a first subset of an oligonucleotide is labeled with one or more detectable labels and a second subset of the identical oligonucleotide—in terms of nucleic acid sequence identity—is unlabeled. In a preferred embodiment the amplification reaction should be performed until the plateau phase of the amplification reaction is reached. This ensures that all of the detectable oligonucleotides are incorporated into the nucleic acids amplification reaction products, i.e. the maximum possible signal strength is reached. Thus, the maximum signal strength is normalized by the predefined ratio of the first labeled subset and the second unlabeled subset of the oligonucleotides that hybridize to the target nucleic acid sequences and thus are incorporated into the nucleic acid amplification reaction products. When the overall amount of detectable labeled oligonucleotides is limited in the amplification reaction, there is a risk that low abundant STR markers may not be detectable in the CE. In order to circumvent this issue, the inventor found out in his experiments that the amplification reaction needs to be performed for a sufficient number of PCR cycles until the plateau phase of the amplification is reached. Therefore, the calculated the ideal number of PCR cycles so that even low abundant STR markers will be amplified until the plateau phase is reached (c.f. example 1).

Herein, the term “plateau phase” means the phase when the maximum possible number of amplification products is reached. Typically an amplification reaction starts with an exponential phase, enters in a linear phase and reaches the plateau phase at higher amplification reaction cycle numbers or—in case of an isothermal amplification—after a certain time of the amplification reaction. One factor that contributes to this plateau phase is the limited availability of the substrate (in particular the oligonucleotides) in the amplification reaction. Typically an amplification reaction, such as a PCR, comprises 30 to 45 amplification cycles depending upon various factors such as the sample conditions, PCR chemistry and PCR itself. For isothermal amplification techniques, the plateau phase can even be reached after less than 60 minutes depending upon the specific assay.

Herein, the term “template nucleic acid” is defined as a nucleic acid molecule that serves as template in a nucleic acid amplification reaction. This can be a DNA molecule, a RNA molecule, a cDNA molecule, a micro RNA molecule or any other amplifiable nucleic acid molecule. The template nucleic acids comprises “target sequences” (i.e. sequences of interest, e.g. STR loci) which are amplified and/or labeled by the use of oligonucleotides which hybridize to said target nucleic acid sequences, to a portion thereof and/or to sequences that flank the target nucleic acid sequences.

Herein, the term “oligonucleotide” is defined as a short polymer comprising three to fifty nucleotides. Oligonucleotides are used as forward primers, revers primers and/or probes in nucleic acid amplification reactions and can be labeled with one or more detectable label.

Nucleic acids can be labeled by intercalating dyes, such as ethidium bromide or SYBR Green, by radioactive phosphates or by fluorescent dyes, also referred to as fluorophores. Labeling oligonucleotides with fluorophores is the most common labeling technique used in molecular biology since it allows for highly sensitive detection and multiplexing in nucleic acid amplification reactions, i.e. the detection of several fluorescence signals at different wavelengths at the same time. Several fluorophores are known to the person skilled in the art. Some examples, but not an exhaustive list, of the fluorophores which might be used in the method according to the invention are: FAM, HEX, Cy5, Cy7, Cy3, ROX, TAMRA, Alexa fluor 488, fluorescein FITC, TAMRA, 6-FAM, BTG, BTR2, BTP, BTY, BTO, LIZ, NED, SID, TAZ, VIC, JOE-6C, TMR-6C, FL-6C, CXR-6C, TOM-6C. The oligonucleotides are usually labeled with one of the above mentioned fluorophores but may also be labeled with more than one of the above mentioned fluorophores. This can be useful in cases when further differentiations are needed in the analysis or in cases where e.g. the sensitivity of the detection signal can be increased by oligonucleotides labeled with more than one label.

Herein the term “detectable label” is defined as one or more fluorophores, radioactive phosphates, biotin, intercalating dyes or any other molecules which can be used for the labeling and detection of nucleic acids.

The oligonucleotides in the methods, kits or compositions according to the invention are either forward primers or reverse primers or probes, forward and revers primers, forward primers and probes, revers primers and probes, forward primers and revers primers and probes or any other oligonucleotides that can be used in nucleic acid amplification reactions or combinations thereof. It is clear to the skilled person in the art that terms like “an oligonucleotide”, “at least one of the hybridizable oligonucleotides”, “a forward primer”, “a revers primer” or “a probe” do not describe just one single oligonucleotide molecule but also a certain amount of oligonucleotide molecules wherein the single molecules of this certain amount of oligonucleotide molecules have the identical sequence. As described, this certain amount of an oligonucleotide may comprise one or more subsets wherein the oligonucleotides are labeled with one or more detectable labels and a subset wherein the oligonucleotides are unlabeled.

Since the signal strength of nucleic acid amplification products, especially in multiplex assays, is dependent on multiple factors such as the chosen fluorophores, the abundance of the template nucleic acids and/or the abundance of the target nucleic acids in the multiplex amplification reaction, the ratio between the first subset of the at least one hybridizable oligonucleotide labeled with one or more detectable labels and the second unlabeled subset of said at least one hybridizable oligonucleotide is of great importance for a proper signal strength normalization according to the invention. Therefore, the inventor investigated and calculated the ratio for different sets of oligonucleotides of different STR loci in multiplex PCR reactions (c.f. example 2).

As a further embodiment of the invention, the method for calculating the optimal ratio of the first subset of the at least one hybridizable oligonucleotide labeled with one or more detectable labels and the second unlabeled subset of said at least one hybridizable oligonucleotide is incorporated herein as follows:

$$\frac{y}{\frac{a}{p}}$$

Wherein y is the overall concentration of each oligonucleotide in the experiment, a is the mean detection signal, e.g. Relative Fluorescence Units (RFU), measured in the CE or any other detection method when 100% of said oligonucleotide are labeled with a detectable Label and p is the RFU which should ideally be measured in said detection method, i.e. the signal strength which is optimal for reliable detection and/or quantification of the nucleic acid amplification products. Usually, p is the mean value or median of a range of signal strengths which is optimal for reliable detection and/or quantification of the nucleic acid amplification products In another embodiment of the invention, the ratio of the first labeled subset and the second unlabeled subset of said at least one hybridizable oligonucleotides is between 100:1 and 1:10000, even more preferably is between 10:1 and 1:5000, even more preferably is between 1:1 and 1:400, even more preferably is between 1:1 and 300, even more preferably is between 1:1 and 200, even more preferably is between 1:1 and 100, even more preferably is between 1:5 and 500, even more preferably between 1:5 and 1:200, even more preferably between 1:5 and 1:100 and most preferably is between 1:10 and 1:100.

Herein, the term “plateau phase” means the phase when the maximum possible number of amplification products is reached. Typically an amplification reaction starts with an exponential phase, enters in a linear phase and reaches the plateau phase at higher PCR cycle numbers or—in case of an isothermal amplification—after a certain time of the amplification reaction. One factor that contributes to this plateau phase is the limited availability of the substrate (in particular the oligonucleotides) in the amplification reaction. Typically a PCR comprises 30 to 45 cycles depending upon various factors such as the sample conditions, PCR chemistry and PCR itself. In isothermal amplification techniques, the plateau phase can even be reached after less than 60 minutes depending upon the specific assay.

Unexpectedly, the inventor investigated that the both, an optimized ratio of the first portion of the oligonucleotide labeled with one or more detectable label and the second unlabeled portion of the oligonucleotide and a number of 45 PCR cycles led to a significant broader range of template nucleic acids concentrations which can be applied to the multiplex PCR assay while also measuring more stable and robust fluorescence signals in the CE.

In a further embodiment of the invention, the amplification reaction in the method according to the invention is performed until the plateau phase of the amplification reaction is reached.

Depending on the specific setup (e.g. sample type, PCR master mix or the like), the plateau phase is reached after 1 to 50 PCR cycles, more preferably after 10 to 50 PCR cycles, even more preferably after 15 to 50 PCR cycles, even more preferably after 25 to 45 PCR cycles, even more preferably after 35-45 PCR cycles and most preferably after 40 to 45 PCR cycles. The amplification reaction may also be an isothermal PCR which runs for a sufficient time until the plateau phase is reached.

Due to the significantly broader range of template nucleic acid concentrations which can be applied to a (multiplex) nucleic acid amplification assay when using the method according to the invention, the steps of quantification, concentration calculation and sample dilution can be avoided. Thus, in yet another embodiment of the invention, the method according to does not require any quantification of the template nucleic acid prior to the amplification reaction or thereafter.

As demonstrated in the comparison experiments (see examples 3 and 4) of the method according to the invention with a commercially available STR kit, a considerably broader range of template nucleic acid was applicable. Therefore, the method according to the invention can be performed when the amount of template nucleic acid in said amplification reaction lies preferably between 1 fg to 5 μg, more preferably between 100 fg to 1 μg, even more preferably between 500 fg to 500 ng, even more preferably between 750 fg to 250 ng, even more preferably between 850 fg to 250 ng, even more preferably between 900 fg to 150 ng, even more preferably between 1 μg to 150ng and most preferably between 2pg to 110 ng.

The signal strength normalized according to the invention may be detected in e.g. capillary electrophoresis, gel electrophoresis, nucleic acid sequencing, next generation sequencing, pyrosequencing, digital PCR, quantitative PCR, real-time PCR, isothermal PCR, microarray analysis or any other method that allows for the detection of labeled nucleic acids.

The amplification reaction according to the invention can be an endpoint PCR, a digital PCR, a real-time PCR, a quantitative PCR, an isothermal PCR, a loop-mediated isothermal amplification (LAMP), a recombinase polymerase amplification (RPA), a nicking enzyme amplification reaction (NEAR), a nicking endonuclease signal amplification (NESA), a rolling circle amplification (RCA), a helicase-dependent amplification (HDA), a hybridization chain reaction (HCR), a multidisplacement amplification, an isothermal assembly reaction or any other nucleic acid amplification reaction or any combination of the above mentioned amplification techniques.

The template nucleic acid can be extracted from a forensic sample containing biological material, sputum, saliva, blood, hairs, hair follicles, sperm, vaginal secretions, liquor, blood plasma, blood serum, fingernails, tissue, urine, plants, microbes, bacteria, viruses, any other parts of the human or animal body or from any other sample containing biological material from which nucleic acids can be extracted. The template nucleic acid can also comprise nucleic acids from several different organisms, individuals and/or sample types. This is for example often the case in forensic samples of sexual assault cases, where both the nucleic acid of the victim and the perpetrator can be amplifiable. Furthermore this is also the case in pooled samples, e.g. pooled blood samples of clinical blood donation services.

In a further embodiment, the methods, composition or kits according to the invention are for use in STR analysis, SNP analysis or genotyping. It may further be applied in nucleic acid library preparation for sequencing applications, next generation sequencing applications, digital PCR applications or microarray applications and in molecular diagnostic applications.

The invention further relates to a composition comprising one or more sets of oligonucleotides, wherein each of said sets of oligonucleotides comprises at least (i) a forward primer, (ii) a revers primer, and/or (iii) a probe or any combination of (i) to (iii) above, and wherein at least one of the oligonucleotides selected from (i) to (iii) above comprises a first subset which is labeled with one or more detectable labels and a second subset which is unlabeled. The composition may further comprise a buffer, a polymerase enzyme, deoxynucleotide triphosphates (dNTPs) or any combination thereof.

In a further embodiment of the composition according to the invention, the ratio of the first labeled subset and the second unlabeled subset of said at least one oligonucleotide is between 100:1 and 1:10000, even more preferably is between 10:1 and 1:5000, even more preferably is between 1:1 and 1:400, even more preferably is between 1:1 and 300, even more preferably is between 1:1 and 200, even more preferably is between 1:1 and 100, even more preferably is between 1:5 and 500, even more preferably between 1:5 and 1:200, even more preferably between 1:5 and 1:100 and most preferably is between 1:10 and 1:100.

The invention further relates to a kit, wherein each of said sets of oligonucleotides comprises at least (i) a forward primer, (ii) a revers primer, and/or (iii) a probe or any combination of (i) to (iii) above, and wherein at least one of the oligonucleotides selected from (i) to (iii) above comprises a first subset which is labeled with one or more detectable labels and a second subset which is unlabeled.

In a further embodiment of the kit according to the invention the ratio of the first labeled subset and the second unlabeled subset of said at least one oligonucleotide is between 100:1 and 1:10000, even more preferably is between 10:1 and 1:5000, even more preferably is between 1:1 and 1:400, even more preferably is between 1:1 and 300, even more preferably is between 1:1 and 200, even more preferably is between 1:1 and 100, even more preferably is between 1:5 and 500, even more preferably between 1:5 and 1:200, even more preferably between 1:5 and 1:100 and most preferably is between 1:10 and 1:100.

The kit according to the invention may comprise one set of oligonucleotides or several sets of nucleotides. In case of molecular diagnostic panels, the kit may comprise up to hundreds or thousands of sets of nucleotides. In preferred embodiment, the kit according to the invention may comprise one or more sets of oligonucleotides for the amplification and labelling of one or more of the loci selected from Amelagonin (AM), TH01, D3S1358, Penta D, D6S1043, D21S11, TPOX, DYS391, D1S1656, D12S391, Penta E, D10S1248, D22S1045, D19S433, D8S1179, D2S1338, D2S441, D18S51, vWA, FGA, D16S539, CSF1PO, D13S317, D5S818, D7S820. Each set of oligonucleotides for the amplification and labeling of Amelagonin (AM), TH01, D3S1358, Penta D, D6S1043 and/or D21S11 comprises a first labeled subset of one or more of the oligonucleotides labeled with 6-FAM and a second unlabeled subset, each set of oligonucleotides for the amplification and labeling of TPOX, DYS391, D1S1656, D12S391 and/or Penta E may comprise a first labeled subset of one or more of the oligonucleotides labeled with BTG and a second unlabeled subset, each set of oligonucleotides for the amplification and labeling of D10S1248, D22S1045, D19S433, D8S1179 and/or D2S1338 may comprise a first labeled subset of one or more of the oligonucleotides labeled with BTY and a second unlabeled subset, each set of oligonucleotides for the amplification and labeling of D2S441, D18S51, vWA, and/or FGA comprises a first labeled subset of one or more of the oligonucleotides labeled with BTR2 and a second unlabeled subset and each set of oligonucleotides for the amplification and labeling of D16S539, CSF1PO, D13S317, D5S818 and/or D7S820 comprises a first labeled subset of one or more of the oligonucleotides labeled with BTP and a second unlabeled subset. In a further embodiment, the above mentioned kit further comprises one or more sets of oligonucleotides for the amplification and labelling of one or more external or internal quality and/or amplification control target sequences. Each set of oligonucleotides for the amplification and labeling of said one or more external or internal quality and/or amplification control target sequences comprises a first labeled subset of one or more of the oligonucleotides labeled with one of the above mentioned labels and a second unlabeled subset. The ratio of the at least first labeled subset of the one or more oligonucleotides and the second unlabeled subset is between 100:1 and 1:10000, even more preferably is between 10:1 and 1:5000, even more preferably is between 1:1 and 1:400, even more preferably is between 1:1 and 300, even more preferably is between 1:1 and 200, even more preferably is between 1:1 and 100, even more preferably is between 1:5 and 500, even more preferably between 1:5 and 1:200, even more preferably between 1:5 and 1:100 and most preferably is between 1:10 and 1:100 (c.f. Example 2) The references cited herein are incorporated by reference in their entirety. The invention has been shown and described with references to preferred embodiments thereof. The invention will be understood by those skilled in the art that various changes in form and details may be made therein without departing from the scope of the invention as defined by the claims.

### EXAMPLES

### Example 1: Calculation of the Number of Cycles in an Amplification Reaction which is Needed to Reach the Plateau Phase of the Amplification Reaction

In order to address both high template nucleic acid concentrations and low template nucleic acid concentrations and/or high or low abundant target nucleic acid sequences, an amplification reaction must be performed until the plateau phase of the amplification reaction is reached. Typically an amplification reaction starts with an exponential phase, enters in a linear phase and reaches the plateau phase at higher amplification reaction cycle numbers. One factor that contributes to this plateau phase is the limited availability of the substrate (e.g. oligonucleotides, polymerase, dNTPs) in the amplification reaction. In this example, the amplification reaction is an end-point PCR. Typically a PCR comprises 30 to 45 cycles depending upon various factors such as the sample conditions, PCR chemistry and the PCR method (e.g. end-point PCR, quantitative PCR). The inventor calculated the theoretical number of PCR cycles which were needed to reach the plateau phase depending on the oligonucleotide concentration. As a starting point, the lowest possible amount of template nucleic acid, i.e. one copy per reaction, and a oligonucleotide concentration of 0.2 μM per reaction was chosen. Furthermore, the inventor assumed a PCR efficiency of 100%, i.e. a doubling of each target nucleic acid sequence in each amplification cycle. The table below shows the calculated quantity of primers in each cycle of an endpoint PCR for up to 45 PCR cycles. It also shows the percentage of oligonucleotides used from the overall amount of oligonucleotides in the PCR after each amplification cycle.

The calculation shows that the whole amount of primers will be used after 43 cycles, i.e. the plateau phase will be reached after 43 cycles based on the primer limitation. The plateau phase is indicated by asterisks in the column “% of oligonucleotides used in 20 μl PCR reactions and a oligonucleotide concentration of 0.2 μM”. For higher amounts of starting template nucleic acids, the plateau phase is reached at correspondingly lower PCR cycles numbers.

| | Quantity of oligonucleotides (forward & revers | % of oligonucleotides used in 20 |
|---|---|---|
| | primer and/or probe) used when starting with | μl PCR reactions and an |
| Number of | lowest possible amount of template nucleic acid | oligonucleotide concentration |
| PCR cycles | (=1 copy/reaction) | of 0.2 μM |
| 1 | 1 | 0.00% |
| 2 | 2 | 0.00% |
| 3 | 4 | 0.00% |
| 4 | 8 | 0.00% |
| 5 | 16 | 0.00% |
| 6 | 32 | 0.00% |
| 7 | 64 | 0.00% |
| 8 | 128 | 0.00% |
| 9 | 256 | 0.00% |
| 10 | 512 | 0.00% |
| 11 | 1024 | 0.00% |
| 12 | 2048 | 0.00% |
| 13 | 4096 | 0.00% |
| 14 | 8192 | 0.00% |
| 15 | 16384 | 0.00% |
| 16 | 32768 | 0.00% |
| 17 | 65536 | 0.00% |
| 18 | 131072 | 0.00% |
| 19 | 262144 | 0.00% |
| 20 | 524288 | 0.00% |
| 21 | 1048576 | 0.00% |
| 22 | 2097152 | 0.00% |
| 23 | 4194304 | 0.00% |
| 24 | 8388608 | 0.00% |
| 25 | 16777216 | 0.00% |
| 26 | 33554432 | 0.00% |
| 27 | 67108864 | 0.00% |
| 28 | 134217728 | 0.01% |
| 29 | 268435456 | 0.01% |
| 30 | 536870912 | 0.02% |
| 31 | 1073741824 | 0.04% |
| 32 | 2147483648 | 0.09% |
| 33 | 4294967296 | 0.18% |
| 34 | 8589934592 | 0.36% |
| 35 | 17179869184 | 0.72% |
| 36 | 34359738368 | 1.43% |
| 37 | 68719476736 | 2.86% |
| 38 | 1.37439E+11 | 5.73% |
| 39 | 2.74878E+11 | 11.45% |
| 40 | 5.49756E+11 | 22.91% |
| 41 | 1.09951E+12 | 45.81% |
| 42 | 2.19902E+12 | 91.63% |
| 43 | 4.39805E+12 | 183.25% * |
| 44 | 8.79609E+12 | 366.50% * |
| 45 | 1.75922E+13 | 733.01% * |

### Example 2: Calculation of the Concentration of the Labeled Subset of an Oligonucleotide

The concentration of the oligonucleotides, wherein at least one of the oligonucleotides comprises a first subset which is labeled with one or more detectable labels and a second subset which is unlabeled was calculated.

In the experiment, the QIAGEN Investigator 26plex QS kit (catalogue number 382615; see: https://www.qiagen.com/us/products/human-id-and-forensics/str-technology/investigator-26plex-qs-kit/) was used. Instead of the primer mix contained therein, the primers for each STR marker were set as 0.1 μM per forward and revers primer, wherein the forward primers were 100% labeled with the fluorescent dye as described in the kit handbook, i.e. no labeled/unlabeled subsets, only labeled forward primers. The PCR cycling protocol was extended to 45 cycles in order to ensure that the plateau phase was reached (c.f. example 1). The injection time in the CE was only 2 seconds instead of 30 seconds as described in the kit handbook since otherwise the RFU of some STR markers would have been too high for detection. All other conditions were as described in the kit handbook.

In independent experiments, template nucleic acid amounts of 16 ng, 8 ng, 4 ng, 2 ng, 1ng and 500 pg were used. As a control for reaching the plateau phase, it was observed that in all experiments nearly the same RFU for each STR marker over all template nucleic acid amounts were measured in the CE, i.e. the whole amount of labeled forward primer was incorporated into the PCR products. The mean RFU measured for each marker over the whole range of template nucleic acid amounts as described above was calculated and is shown in the table below.

The mean RFU was then used to calculate the concentration of the first portion of the forward primer which is labeled according to the invention. The calculation was based on the goal to measure a RFU of about 4000 in the CE which is in the ideal range for optimal peak detection. Based on the results, the concentration of the first labeled subset of the oligonucleotides (here: the forward primers) was calculated as follows:

$$\frac{0.1}{\frac{a \times 15}{4000}}$$

wherein 0.1 is the concentration of the labeled forward primers in the experiment, a is the mean RFU measure in the CE with 100% labeled forward primers and 4000 is the RFU which should ideally be measured in the CE. The mean RFU measured in the CE is multiplied by 15 since 30 seconds is the optimal injection time in the CE according to the kit handbook. The lowest possible concentration of the first portion of the forward primer labeled with one or more detectable label was set as 0.001 μM. The sum of the first portion of the forward primer labeled with one or more detectable label and the second unlabeled portion of the forward primer were 0.1 μM. The calculated concentrations are shown in the table below.

| | Mean RFU measured | | | |
|---|---|---|---|---|
| | in capillary | Concentration of | | |
| | electrophoresis after | labeled forward | | |
| | PCR with 100% | primers [μM] to | Concentration | |
| | labeled forward and | measure about 4000 | of unlabeled | Concentration of |
| STR | unlabeled reverse | RFU in capillary | forward primers | unlabeled revers |
| Marker | primers | electrophoresis | [μM] | primers [μM] |
| AM | 30036 | 0.001 | 0.099 | 0.1 |
| TH01 | 23053 | 0.001 | 0.099 | 0.1 |
| D3S1358 | 20902 | 0.001 | 0.099 | 0.1 |
| Penta D | 23729 | 0.001 | 0.099 | 0.1 |
| D6S1043 | 3362 | 0.008 | 0.092 | 0.1 |
| D21S11 | 5365 | 0.005 | 0.095 | 0.1 |
| TPOX | 26860 | 0.001 | 0.099 | 0.1 |
| DYS391 | 30334 | 0.001 | 0.099 | 0.1 |
| D1S1656 | 25653 | 0.001 | 0.099 | 0.1 |
| D12S391 | 24258 | 0.001 | 0.099 | 0.1 |
| Penta E | 2904 | 0.010 | 0.090 | 0.1 |
| D10S1248 | 4554 | 0.006 | 0.094 | 0.1 |
| D22S1045 | 7068 | 0.004 | 0.096 | 0.1 |
| D19S433 | 6144 | 0.004 | 0.096 | 0.1 |
| D8S1179 | 8061 | 0.003 | 0.097 | 0.1 |
| D2S1338 | 4291 | 0.006 | 0.094 | 0.1 |
| D2S441 | 4004 | 0.007 | 0.093 | 0.1 |
| D18S51 | 6737 | 0.004 | 0.096 | 0.1 |
| vWA | 2800 | 0.010 | 0.090 | 0.1 |
| FGA | 4389 | 0.006 | 0.094 | 0.1 |
| D16S539 | 9017 | 0.003 | 0.097 | 0.1 |
| CSF1PO | 7543 | 0.004 | 0.096 | 0.1 |
| D13S317 | 7941 | 0.003 | 0.097 | 0.1 |
| D5S818 | 8910 | 0.003 | 0.097 | 0.1 |
| D7S820 | 6489 | 0.004 | 0.096 | 0.1 |

### Example 3: Comparison of the Performance of the Invention with a Commercially Available STIR Analysis Kit

In order to investigate the performance of the primer mix that was developed as described in example 2 (referred to as “normalized”), comparison experiments between a commercially available forensic STR analysis kit and the same kit wherein the primer mix was replaced by the new normalized primer mix according to example 2 over a wide range of template DNA amounts were conducted.

The commercially available forensic STR analysis kit was QIAGEN Investigator 26plex QS kit (catalogue number 382615; see: https://www.qiagen.com/us/products/human-id-and-forensics/str-technology/investigator-26plex-qs-kit/) used as reference/coinparison kit. In both, the not normalized comparison experiments (i.e. above mentioned commercially available kit) and the normalized experiments (i.e. above mentioned kit with the new primer mix), template DNA amounts of 16 ng (FIG. 2a), 8 ng (FIG. 2b), 500 pg (FIG. 2c), 124 pg (FIG. 2d), 32 pg (FIG. 2e), 8 pg (FIG. 2f) and 2 pg (FIG. 2g) were used. As general assay control, non-template controls (NTC) were included in all experiments.

Despite the replacement of the primer mix by the new normalized primer mix and the prolonged PCR with 45 cycles in the normalized experiments, all experiments were performed as described in the kit handbook.

While the inventor expected to fail to obtain the STR profiles in the not normalized experiments with the highest amounts of template DNA due to capillary overload or fluorescence signals far above the detection range in the CE, he unexpectedly was able to obtain the full STR profiles in the normalized experiments (FIG. 2 a and FIG. 2b). According to the kit handbook, 500 pg template DNA is the optimum amount for the assay. The results of the corresponding experiments (FIG. 2c) showed that in both the not normalized and the normalized experiments the full STR profile could be obtained. The same is true for 124 pg template DNA (FIG. 2d), however, the measured RFU already decreased dramatically in the not normalized experiment compared to the RFU measured in the experiment with 500 pg template DNA. This is also observed in the experiments with the lowest amounts of template DNA (FIG. 2e to FIG. 2f). In the not normalized experiments the electropherogram peaks were nearly not detectable due to extremely low RFU, the RFU in the normalized experiments were mainly well detectable due to the higher PCR cycle number. In FIG. 3 the findings of all these experiments as described above are summarized and the percentages of the STR profiles that could be obtained are indicated.

This clearly shows the improvement of the invention over the prior art.

### Example 4: Comparison Between State of the Art Method and the Method, Composition and Kit According to the Invention

To further demonstrate the superiority of the invention over the prior art, additional comparison examples were performed. A dilution series of template DNA with theoretical concentrations between 8.53333 to 0.00013 ng/μl was prepared. The concentration was quantified with the QIAGEN Investigator Quantiplex Pro Kit (catalogue number 387216; see https://www.qiagen.com/se/shop/new-products/investigator-quantiplex-pro-kit/) as usually necessary in the STR analysis workflow. Quantified concentrations of the dilution series were between 7.31074 to 0.00027 ng/μl (c.f. table below). All experiments were performed as described in above mentioned kit handbook (quantification) and the subsequent STR analysis was performed as described in example 3 with the QIAGEN Investigator 26plex QS kit (catalogue number 382615; see: https://www.qiagen.com/us/products/human-id-and-forensics/str-technology/investigator-26plex-qs-kit/). The normalized experiments were also performed as described in example 3 (new primer mix, 45 PCR cycles).

As shown in the table below, sample 1 to 4 required further dilution before a concentration according to the kit handbook was available for STIR analysis, samples 5 to 8 were in a concentration range which did not require further dilution, samples 9 to 15 were below the template DNA concentration as recommended in the kit handbook.

| | | | Amount of | | |
|---|---|---|---|---|---|
| | | | template DNA in | Q Inv. 26plex | |
| | Theoretical | Quantified | the PCR (rounded | QS | |
| | concentration | concentration | to the nearest full | μl for | |
| Sample | [ng/μl] | [ng/μl] | number] | 500 pg/reaction | Comment |
| 1 | 8.53333 | 7.31074 | 110 ng | 0.07 | Predilution step |
| 2 | 4.26667 | 4.14310 | 62 ng | 0.12 | required |
| 3 | 2.13333 | 1.90972 | 29 ng | 0.26 | |
| 4 | 1.06667 | 0.85320 | 13 ng | 0.59 | |
| 5 | 0.53333 | 0.46251 | 7 ng | 1.08 | No predilution step |
| 6 | 0.26667 | 0.24188 | 4 ng | 2.07 | required |
| 7 | 0.13333 | 0.12173 | 2 ng | 4.11 | |
| 8 | 0.06667 | 0.05285 | 793 pg | 9.46 | |
| 9 | 0.03333 | 0.02754 | 413 pg | 18.16 | Max. input volume |
| 10 | 0.00827 | 0.00676 | 101 pg | 73.94 | 15 μl. Decreasing |
| 11 | 0.00213 | 0.00143 | 21 pg | 348.71 | RFU will be |
| 12 | 0.00107 | 0.00068 | 10 pg | 734.15 | measured which |
| 13 | 0.00053 | 0.00037 | 6 pg | 1340.04 | may lead to errors |
| 14 | 0.00027 | 0.00017 | 3 pg | 2954.10 | or failing to |
| 15 | 0.00013 | 0.00027 | 4 pg | 1863.45 | determine STR |
| NTC | 0 | 0 | 0 pg | — | peaks. |

In contrast, samples 1 to 15 were directly used in the normalized experiments according to the invention without any further dilution steps. All experiments were performed with an input volume of 15 μl of the template DNA solutions as recommended in the kit handbook.

Without any dilution, normalized samples showed similar, perfectly analyzable STIR profiles as in the prediluted not normalized samples (FIG. 4a-FIG. 4d). Despite the higher number of PCR cycles, normalized experiments with the optimal PCR concentrations according to the kit handbook were also perfectly analyzable (FIG. 4e-FIG. 4h). While in the not normalized experiments the RFU decreased dramatically at lower template DNA concentrations and it was not possible to obtain the STIR profiles anymore, the RFU in the normalized experiments was still high and almost the full STIR profiles could be analyzed (FIG. 4i-FIG. 4o).

These experiments clearly show that it the method, composition and kit according to the invention allow for a broader range of template nucleic acid concentrations and thus to avoid the quantification and dilution steps.

### DESCRIPTION OF THE FIGURES

FIG. 1
a) Electropherogram of a perfectly analyzable STR profile. All STR alleles are detectable with distinct peaks which are in a RFU range which is not too high (no capillary overload) and not too low. This electropherogram was obtained with the QIAGEN Investigator 26plex QS kit after quantification of the DNA concentration of the sample with the QIAGEN Investigator Quantiplex Pro Kit. Like for other commercially available STR analysis kits, the sample was diluted and the ideal amount of 0.5 ng DNA was used in the experiment.
b) Example of an electropherogram with too high template DNA concentration. In order to determine the STR fragment sizes, size standards are included in all CE runs. In case of too high sample input (e.g. without quantification/dilution of the sample), capillaries are overloaded which leads to fluorescence signals beyond the detection range which interfere with the size standard channel and the STR profile cannot be determined.
c) Example of an STR analysis in which the input sample had a too low DNA concentration which resulted in low RFU values and only a few STR alleles were detectable.

FIG. 2

Relative Fluorescence Units (RFU) for each STR marker of the QIAGEN Investigator 26plex QS kit are shown for experiments with different template DNA amounts as described in example 3:
a) 16 ng template DNA
b) 8 ng template DNA
c) 500 pg template DNA
d) 124 pg template DNA
e) 32 pg template DNA
f) 8 pg template DNA
g) 2 pg template DNA

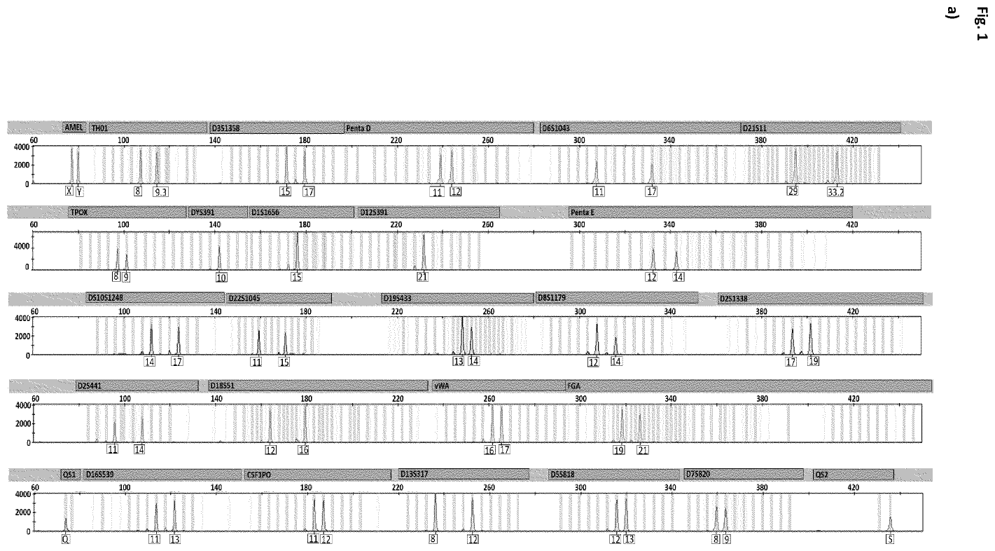

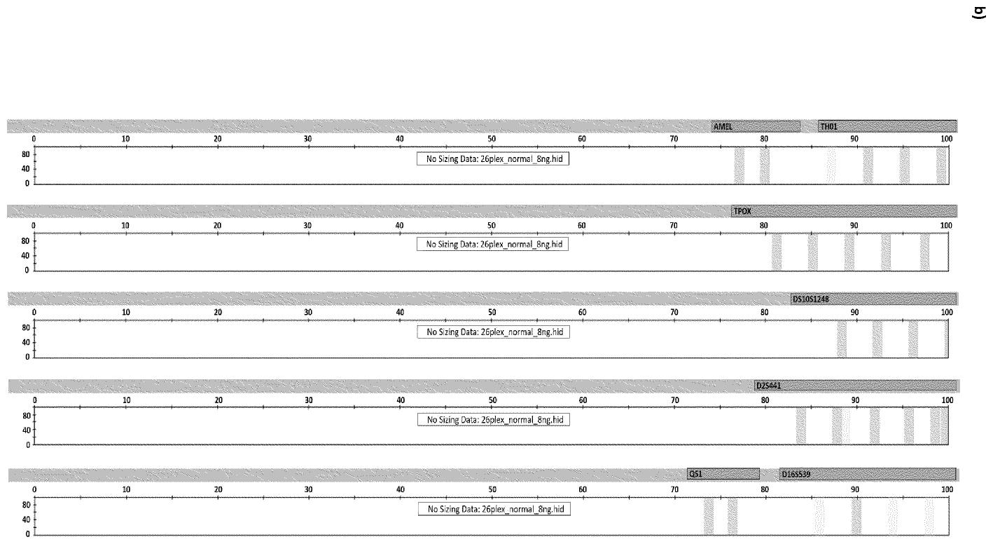

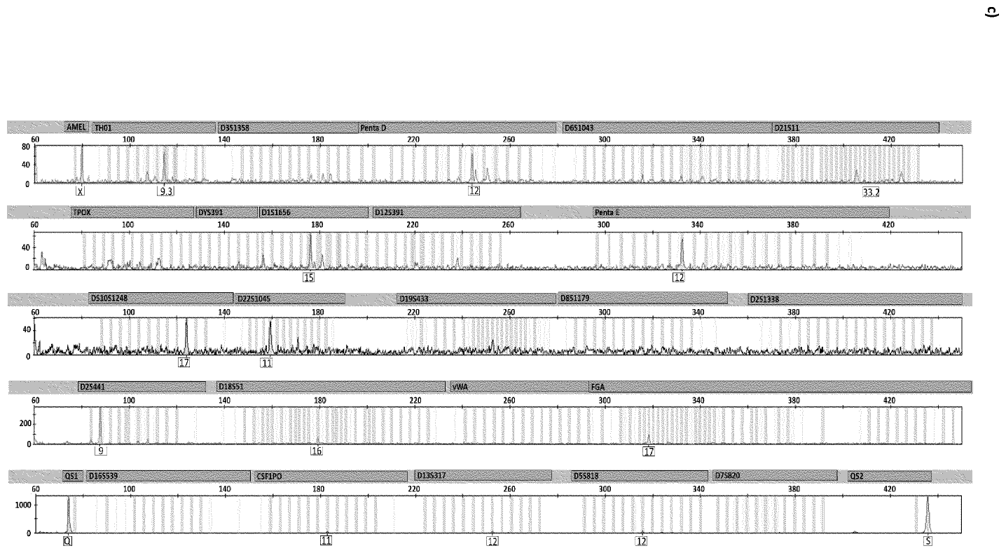

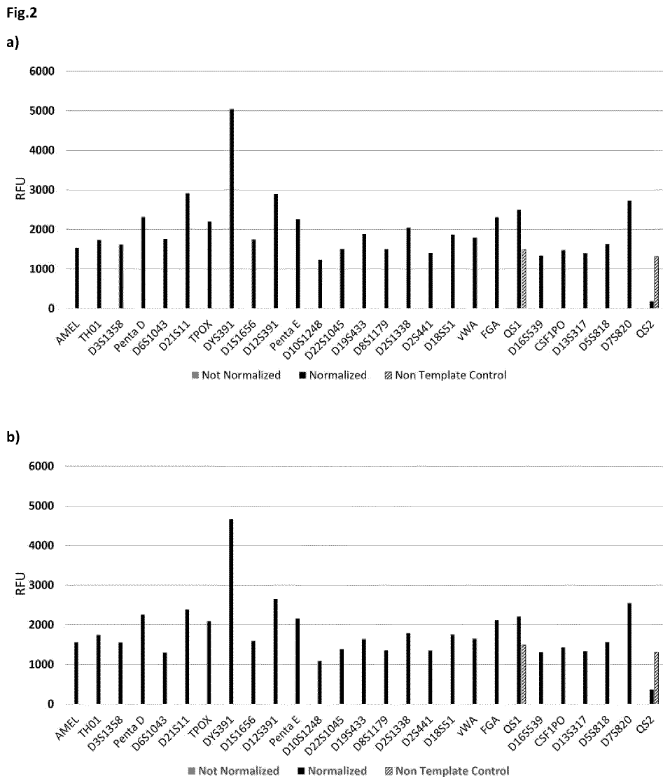

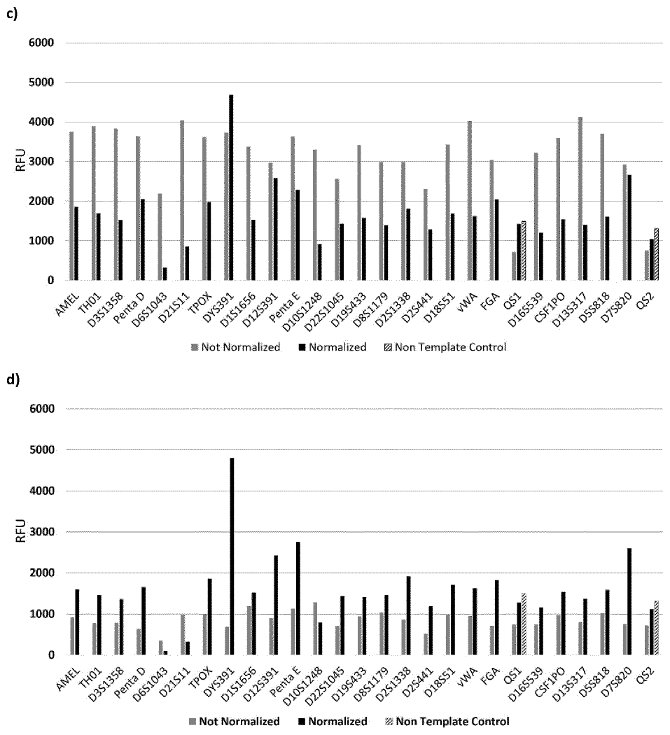

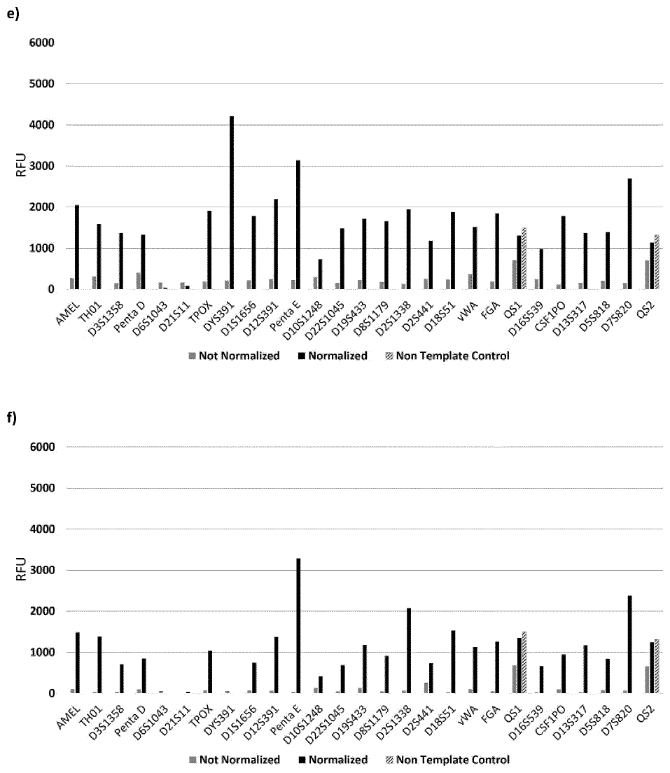

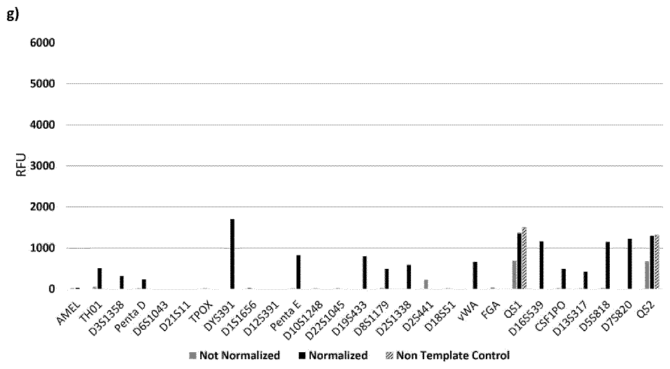

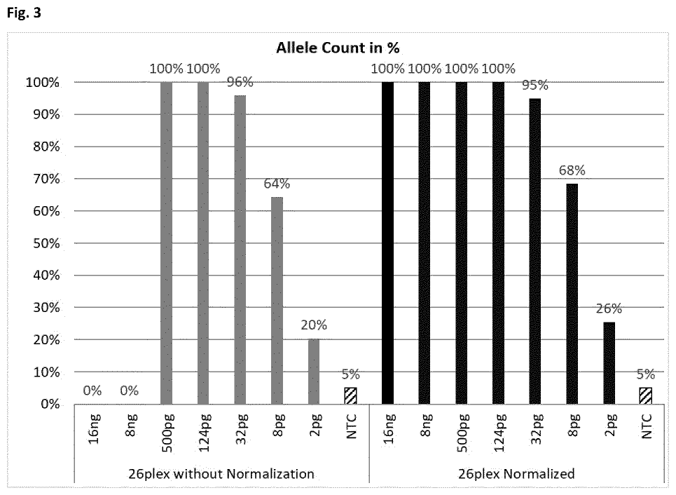

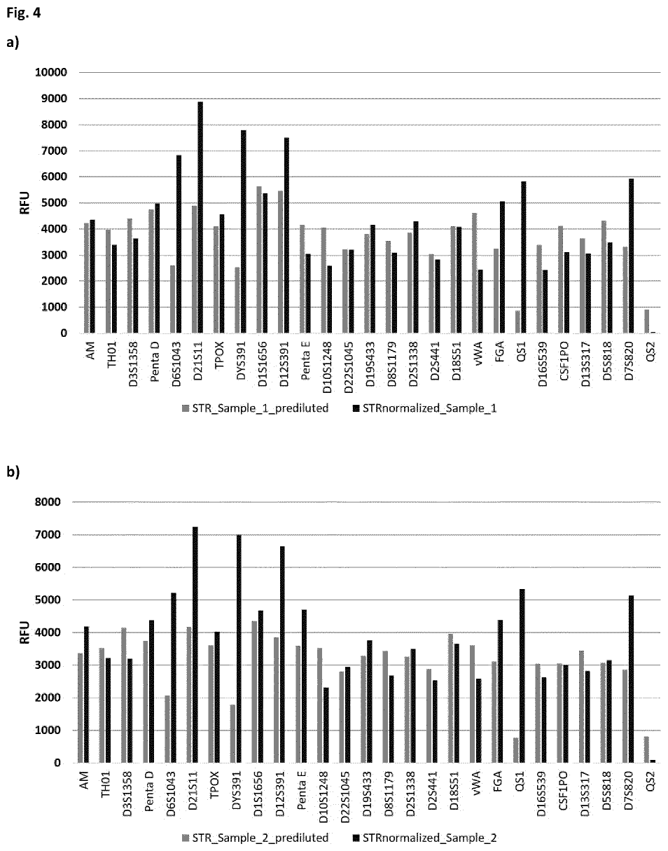

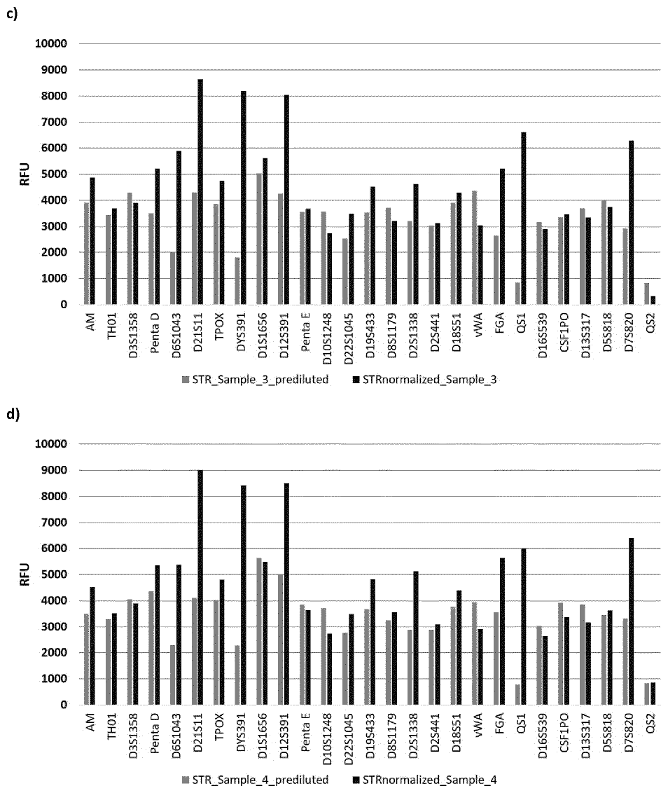

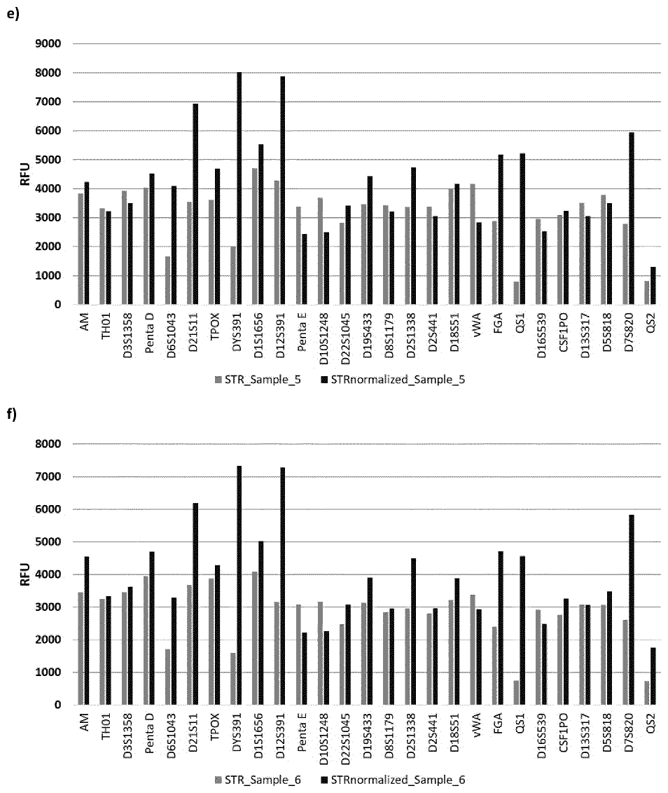

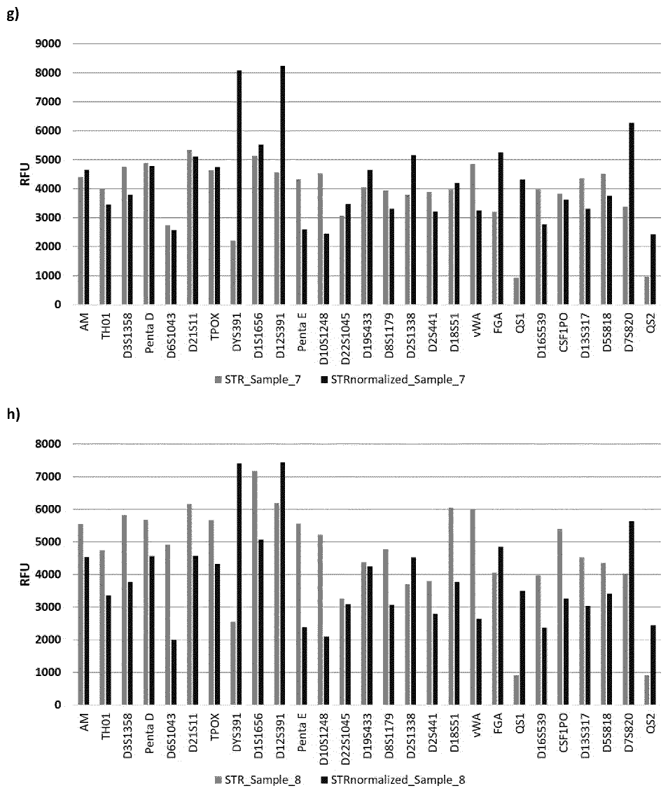

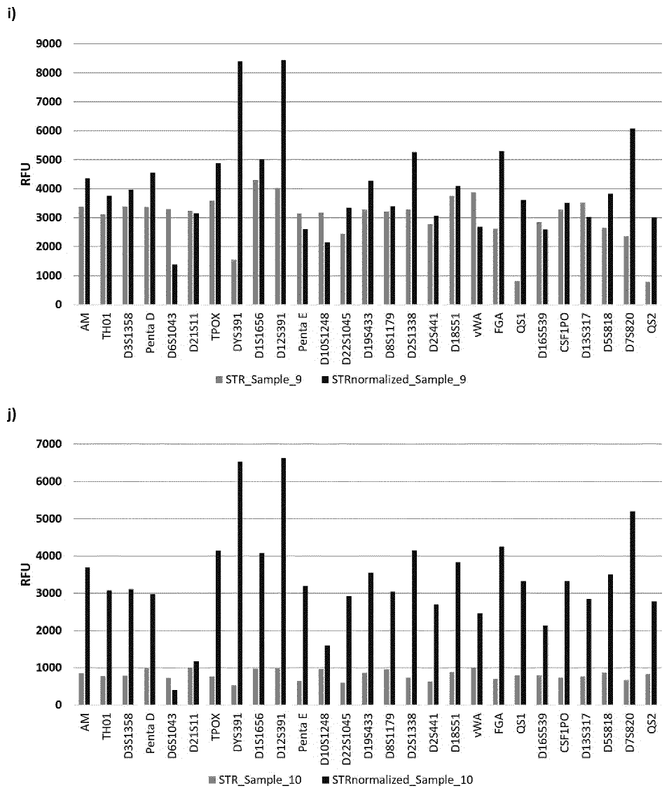

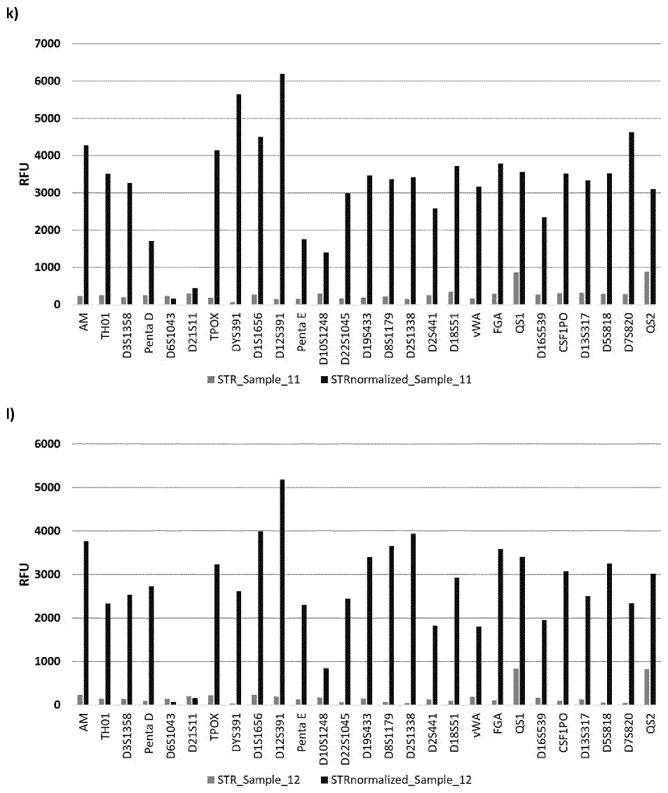

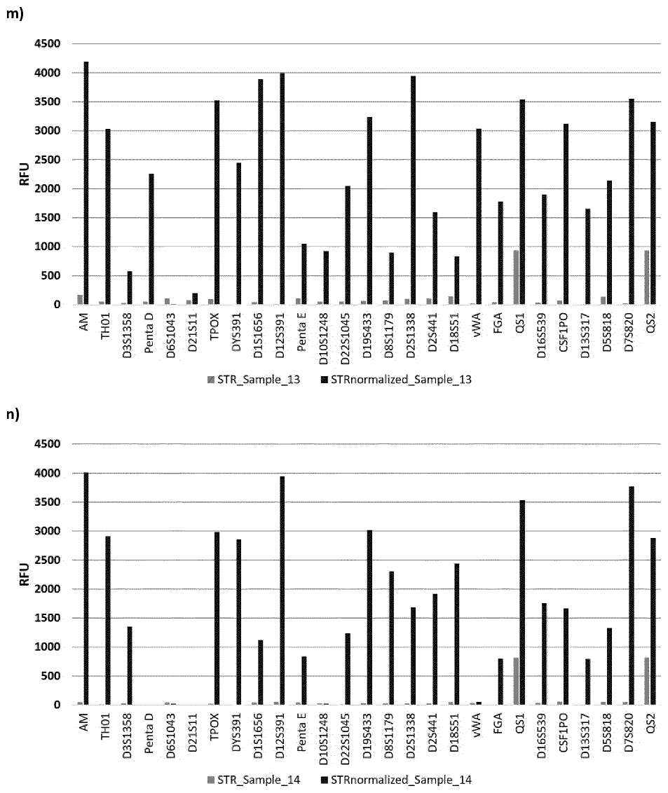

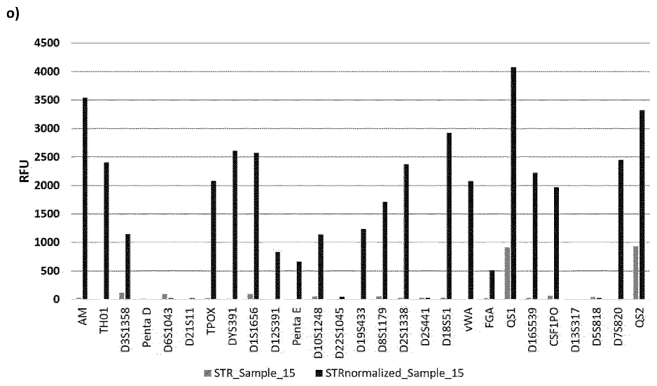

Grey bars show the RFU measured for the not normalized experiments and black bars show the RFU measured for the normalized experiments according to the invention. Due to too high template DNA amounts, no STR markers were detectable in FIG. 2a) and FIG. 2b) for the not normalized experiments and considerable more STR markers were also not detectable for low template DNA amounts in the not normalized experiments compared to the normalized experiments (FIG. 2e to FIG. 2g).

FIG. 3

Summary of all experiments described in example 3. The figure shows the percentage of the allele count (i.e. the percentage of the STR profile which was analyzable) of the STR profiles at different template DNA amounts. In the normalized experiments according to the invention, full STR profiles were obtained even for the high template DNA amounts while the STR profiles were also more complete for very low template DNA amounts. In comparison, in the not normalized STR assay according to the prior art it was not possible to obtain an analyzable STR profile for the high template DNA amounts and for the very low template DNA amounts the STR profiles were also more incomplete than in the normalized experiments according to the invention.

FIG. 4

Comparison of the invention with a STR analysis kit of the prior art (QIAGEN Investigator 26plex QS kit) over a broad range of template DNA concentrations/amounts. Samples were quantified with QIAGEN Investigator Quantiplex Pro Kit. Predilution was necessary for samples 1 to 4 in the not normalized experiments while this was not necessary for the normalized experiments. Template DNA concentrations of samples 9 to 15 were below the recommended concentrations according to the kit handbook and it was not possible to obtain full STR profiles in the experiments with the lowest template DNA concentration. Grey bars show the RFU measured for the not normalized experiments and black bars show the RFU measured for the normalized experiments according to the invention.
a) 7.31074 ng/μl (predilution necessary in not normalized experiments)
b) 4.14310 ng/μl (predilution necessary in not normalized experiments)
c) 1.90972 ng/μl (predilution necessary in not normalized experiments)
d) 0.85320 ng/μl (predilution necessary in not normalized experiments)
e) 0.46251 ng/μl
f) 0.24188 ng/μl
g) 0.12173 ng/μl
h) 0.05285 ng/μl
i) 0.02754 ng/μl (concentration below the concentration range as referred to in the kit handbook)
j) 0.00676 ng/μl (concentration below the concentration range as referred to in the kit handbook)
k) 0.00143 ng/μl (concentration below the concentration range as referred to in the kit handbook)
l) 0.00068 ng/μl (concentration below the concentration range as referred to in the kit handbook)
m) 0.00037 ng/μl (concentration below the concentration range as referred to in the kit handbook)
n) 0.00017 ng/μl (concentration below the concentration range as referred to in the kit handbook)
o) 0.00027 ng/μl (concentration below the concentration range as referred to in the kit handbook)

1. Multiplex nucleic acid amplification reaction comprising at least two separate amplicons, with normalized signal strength, wherein
for at least one amplicon either the forward primer oligonucleotides, or
the reverse primers oligonucleotides, or the optional detection probe oligonucleotides, are only partially labeled, and
the subset amount of labeled primer/probe oligonucleotides to subset amount of unlabeled primer/probe oligonucleotides is adjusted so that the signal strength the at least two amplicons is equal or nearly equal,
wherein the amplification reaction reaches the plateau phase.

2. Multiplex nucleic acid amplification reaction according to claim 1, wherein the ratio of the labeled subset to unlabeled subset is between 100:1 and 1:10000, even more preferably is between 10:1 and 1:5000, even more preferably is between 1:1 and 1:400, even more preferably is between 1:1 and 300, even more preferably is between 1:1 and 200, even more preferably is between 1:1 and 100, even more preferably is between 1:5 and 500, even more preferably between 1:5 and 1:200, even more preferably between 1:5 and 1:100 and most preferably is between 1:10 and 1:100.

3. Reaction according to any of the preceding claims, wherein the amplicons are located on one or more template nucleic acids and the amount of the one or more template nucleic acids in said nucleic acid amplification reaction mixture is between 1 fg to 5 μg, more preferably between 100 fg to 1 μg, even more preferably between 500 fg to 500 ng, even more preferably between 750 fg to 250 ng, even more preferably between 850 fg to 250 ng, even more preferably between 900 fg to 150 ng, even more preferably between 1 μg to 150 ng and most preferably between 2 μg to 110 ng.

4. Reaction according to claim 3, wherein no quantification and/or dilution of the one or more template nucleic acids is performed prior to the amplification reaction.

5. Reaction according to any of the preceding claims, wherein detecting the labels of the first subset of oligonucleotides is performed in capillary electrophoresis, gel electrophoresis, pyrosequencing, sanger sequencing, next generation sequencing, digital PCR, real-time PCR, quantitative PCR, isothermal PCR, or in microarray analysis.

6. Reaction according to any of the preceding claims, wherein the reaction is an endpoint PCR, a digital PCR, a real-time PCR, a quantitative PCR, an isothermal PCR, a loop-mediated isothermal amplification, a recombinase polymerase amplification, a nicking enzyme amplification reaction, a nicking endonuclease signal amplification, a rolling circle amplification, a helicase-dependent amplification, a hybridization chain reaction, a multidisplacement amplification, an isothermal assembly reaction or any combination thereof.

7. Reaction according to claims 3 to 6, wherein the one or more template nucleic acids are extracted from a forensic sample containing biological material, sputum, saliva, blood, hairs, hair follicles, sperm, vaginal secretions, liquor, blood plasma, blood serum, fingernails, tissue, urine, plants, microbes, bacteria, viruses, any other parts of the human or animal body or from any other sample containing biological material from which nucleic acids can be extracted.

8. Reaction according to any of the preceding claims for use in STR analysis, SNP analysis, genotyping, molecular diagnostics, genetic research or population genetics.

9. A composition for using in the reaction according to any of the claims 1 to 8, the composition comprising two or more sets of oligonucleotides, wherein each of said sets of oligonucleotides comprises at least
(i) a forward primer,
(ii) a revers primer, and/or
(iii) a probe, and
wherein at least one of the oligonucleotides selected from (i) to (iii) above comprises a first subset which is labeled with one or more detectable labels and a second subset which is unlabeled.

10. The composition of claim 9, wherein the ratio of the first labeled subset and the second unlabeled subset of said at least one of the oligonucleotides is between 100:1 and 1:10000, even more preferably is between 10:1 and 1:5000, even more preferably is between 1:1 and 1:400, even more preferably is between 1:1 and 300, even more preferably is between 1:1 and 200, even more preferably is between 1:1 and 100, even more preferably is between 1:5 and 500, even more preferably between 1:5 and 1:200, even more preferably between 1:5 and 1:100 and most preferably is between 1:10 and 1:100.

11. A kit for using in the reaction according to any of the claims 1 to 8, the kit comprising one or more sets of oligonucleotides, wherein each of said sets of oligonucleotides comprises at least
(i) a forward primer,
(ii) a revers primer, and optionally
(iii) a probe, and
wherein at least one of the oligonucleotides selected from (i) to (iii) above comprises a first subset of oligonucleotides which is labeled with one or more detectable labels and a second subset of oligonucleotides which is unlabeled.

12. The kit according to claim 11, wherein the ratio of the first labeled subset and the second unlabeled subset of oligonucleotides is between 100:1 and 1:10000, even more preferably is between 10:1 and 1:5000, even more preferably is between 1:1 and 1:400, even more preferably is between 1:1 and 300, even more preferably is between 1:1 and 200, even more preferably is between 1:1 and 100, even more preferably is between 1:5 and 500, even more preferably between 1:5 and 1:200, even more preferably between 1:5 and 1:100 and most preferably is between 1:10 and 1:100.
